SEQUENCE LISTING (2 sequences)

SEQ ID NO: 1 - protein, 706 aa, Homo sapiens
  PEPTID (1)..(706)
MSPFLRIGLSNFDCGSCQSCQGEAVNPYCAVLVKEYVESENGQMYIQKKPTMYPPWDSTFDAHINKGRVMQIIVKGKNVDLISETTVELYSLAERCRKNNGKTEIWLELKPQGRMLMNARYFLEMSDTKDMNEFETEGFFALHQRRGAIKQAKVHHVKCHEFTATFFPQPTFCSVCHEFVWGLNKQGYQCRQCNAAIHKKCIDKVIAKCTGSAINSRETMFHKERFKIDMPHRFKVYNYKSPTFCEHCGTLLWGLARQGLKCDACGMNVHHRCQTKVANLCGINQKLMAEALAMIESTQQARCLRDTEQIFREGPVEIGLPCSIKNEARPPCLPTPGKREPQGISWESPLDEVDKMCHLPEPELNKERPSLQIKLKIEDFILHKMLGKGSFGKVFLAEFKKTNQFFAIKALKKDVVLMDDDVECTMVEKRVLSLAWEHPFLTHMFCTFQTKENLFFVMEYLNGGDLMYHIQSCHKFDLSRATFYAAEIILGLQFLHSKGIVYRDLKLDNILLDKDGHIKIADFGMCKENMLGDAKTNTFCGTPDYIAPEILLGQKYNHSVDWWSFGVLLYEMLIGQSPFHGQDEEELFHSIRMDNPFYPRWLEKEAKDLLVKLFVREPEKRLGVRGDIRQHPLFREINWEELERKEIDPPFRPKVKSPFDCSNFDKEFLNEKPRLSFADRALINSMDQNMFRNFSFMNPGMERLIS

SEQ ID NO: 2 - protein, 37 aa, Homo sapiens
  MOD_RES (19)..(19): PHOSPHORYLATION
CIDKVIAKCTGSAINSRETMFHKERFKIDMPHRFKVY

(19) United States
(12) Patent Application Publication
(10) Pub. No.: US 2009/0170125 A1
(43) Pub. Date: Jul. 2, 2009

(54) Cellular assay method for identifying pkc-0 inhibitors

(75) Inventors: Isabelle Heit, Hüttwilen (CH); Monika Baudler, Konstanz (DE); Sascha Dammeier, Konstanz (DE); Michaela Schäfer, Orsingen-Nenzingen (DE); Hubert Paul, Wielenbach (DE); Gottfried Baier, Innsbruck (AT); Stephen Shaw, Bethesda, MD (US)

Correspondence Address: NATH & ASSOCIATES PLLC, 112 South West Street, Alexandria, VA, 22314, US

(73) Assignee: NYCOMED GMBH, KONSTANZ (DE)

(21) Appl. No.: 11/919,178

(22) PCT Filed: Apr. 27, 2006
(86) PCT No.: PCT/EP2006/061891

§ 371 Date: Jan. 25, 2008

(30) Foreign Application Priority Data
May 2, 2005 (DE) 102005020754.5

(51) Int. Cl. C12Q 1/48; G01N 33/573; C07K 16/40

(52) U.S. Cl. 435/7.4; 435/15; 435/7.92; 530/387.1; 530/387.9

(57) ABSTRACT

The invention relates to a method for investigating the modulating effect of a test substance on a PKCθ-dependent signal transduction pathway or for finding a PKCθ modulator in a human or animal cell, including the steps
(a) contacting the cell with the test substance or with the PKCθ modulator;
(b) where appropriate inducing the kinase activity of PKCθ;
(c) incubating the cell under conditions which bring about phosphorylation at least of a serine or threonine residue of PKCθ;
(d) where appropriate lysing the cell; and
(e) determining the phosphorylation content of the at least one serine or threonine residue of PKCθ.

The invention relates to a method for investigating the modulating effect of test substances on a PKCθ-dependent signal transducton pathway and for finding PKCθ modulators in a human or animal cell. In a preferred embodiment, the method is suitable for determining the modulating effect of test substances on the kinase activity of isoform θ of protein kinase C (PKCθ).

Protein kinase C (PKC) is involved in many signal transduction processes and in the regulation of proliferation and differentiation. Isoform θ of protein kinase C (PKCθ) is one of the key enzymes in signal transduction in T cells and thus plays an important part in the cell-mediated immune response.

The activation of T cells takes place by a complex mechanism in which a plurality of enzymes and receptors are involved. The activation is initiated by stimulation of T-cell receptor-coupled tyrosine kinases of the Src and Syk families, which phosphorylate different cellular substrates. This is followed by the formation of membrane-signal complexes which are involved in various signal transduction cascades. These transmit signals to the cell nucleus and there induce various genetic processes.

PKCθ is an isoform of the PKC family whose kinase activity depends on diacylglycerol but not on Ca²⁺. PKCθ is expressed substantially selectively in skeletal muscle cells and T cells (T lymphocytes) and plays a central part in the activation of T cells. PKCθ specifically activates the c-Jun N-terminal kinase (JNK) and the transcription factor AP-1 in T cells, and acts synergistically together with calcineurin in the activation of the IL-2 gene. In addition, PKCθ is the only protein kinase C isoform known to date to be involved in the formation of a membrane-signal complex when the T cell comes into contact with a stimulator cell. Two isoforms of PKCθ are known, PKCθI and PKCθII, of which the latter possibly plays a part in spermatogenesis (cf. Y. S. Niino et al., J. Biol. Chem. 2001, 276(39), 36711).

PKCθ is a good target in the search for novel pharmacological active ingredients such as novel immunomodulators, especially immunostimulants and immunosuppressants, or agents for treating muscle disorders.

Methods for identifying agents which have an effect on the phosphorylation of PKCθ are known in the art. Thus, WO 01/48236 discloses that PKCθ is phosphorylated on Tyr⁹⁰ in the regulatory domain in T cells of the Jurkat cell line by the tyrosine kinase Lck, a member of the Src family, as a result of TCR/CD3 activation. It is proposed to identify inhibitors of the tyrosine kinase Lck by measuring their effect on the tyrosine phosphorylation of PKCθ in Jurkat T cells after TCR/CD3 activation.

Besides Tyr⁹⁰, other phosphorylation sites of PKCθ have been described in the art, namely Thr⁵³⁸, Ser⁶⁷⁶ and Ser⁶⁹⁵ (cf. Y. Liu et al., Biochem. J. (2002) 361, 255-265). Phospho-specific antibodies against these phosphorylation sites are now also commercially available (anti-PKCθ-phospho-Thr⁵³⁸, anti-PKCθ-phospho-Ser676 and anti-PKCθ-phospho-Ser⁶⁹⁵ antibodies), e.g. from abcam Ltd., Cambridge, UK; Cell Signalling Technology Inc., Beverly, USA; BioSource International, Camarillo, USA; Santa Cruz Biotechnology, Santa Cruz, USA; and Novus Biologicals, Inc., Littleton, USA.

Conventional test systems for determining the enzymic activity of PKCθ are normally based on an enzymatic in vitro substrate phosphorylation assay in which recombinantly expressed protein is used. However, unlike the situation in vivo, where the enzyme must first be activated via a cascade, the enzyme provided in these test systems is already active and therefore does not correspond to its state under physiological conditions. The result of this is that, for example, membrane interactions and interactions with other proteins involved in the signal transduction cascade, such as, for example, a possible binding to adaptor proteins, cannot be detected by conventional test systems.

The invention is thus based on the object of providing a test system in which an investigation of the modulating effect of a test substance on a PKCθ-dependent signal transduction pathway, especially on the enzymic activity of PKCθ, is possible, or a PKCθ modulator can be found, under in vivo conditions, i.e. with PKCθ as physiological substrate. The test system was intended to be sensitive and, if possible, suitable for high-throughput screening (HTS) of test substance libraries. It was intended to make the recording of dose-effect relationships possible.

It has surprisingly been found that this object can be achieved by a method
for investigating the modulating effect of a test substance on a PKCθ-dependent signal transduction pathway or
for finding a PKCθ modulator
   in a human or animal cell, including the steps
(a) contacting the cell with the test substance or with the PKCθ modulator;
(b) where appropriate inducing the kinase activity of PKCθ;
(c) incubating the cell under conditions which bring about phosphorylation at least of a serine or threonine residue of PKCθ, preferably phosphorylation of the threonine residue in position 219 of PKCθ;
(d) where appropriate lysing the cell; and
(e) determining the phosphorylation content of the at least one serine or threonine residue of PKCθ, preferably of the threonine residue in position 219 of PKCθ.

A “test substance having a modulating effect” on a PKCθ-dependent signal transduction pathway in the context of the description comprises a substance which has an activating or inhibiting effect on a signal transduction pathway in which PKCθ is involved, i.e. within which PKCθ catalyses a reaction which is to proceed. The modulating, i.e. activating or inhibiting, effect of the test substance is preferably manifested by formation of a final product or intermediate within the signal transduction pathway to an enhanced or reduced extent in vivo in the presence of the test substance, relative to the situation in the absence of this test substance under conditions which are otherwise the same. This final product or intermediate is moreover preferably formed within the signal transduction pathway after PKCθ has already fulfilled its function. This final product or intermediate is preferably the direct reaction product of the phosphorylation reaction which is catalysed by PKCθ. The modulating effect of the test substance is, however, preferably also manifested in the following products which are eventually derived, where appropriate with involvement of further enzymes, from this direct reaction product in vivo.

A “PKCθ-dependent signal transduction pathway” in the context of the description is in principle any biochemical reaction pathway in which PKCθ is involved, preferably an enzyme cascade. It is possible in this connection for PKCθ in turn to be the substrate of a particular reaction, for example a phosphorylation reaction, in which case the test substance displays a direct or indirect effect on the rate of this phosphorylation reaction. It is preferred for the test substance to display its modulating effect on a phosphorylation reaction which is catalysed by PKCθ itself. The test substance preferably displays its modulating effect on a PKCθ-catalysed phosphorylation reaction where PKCθ is itself the substrate of this reaction (autophosphorylation).

The test substance need not in this case act directly on PKCθ. On the contrary, it is also possible for example for certain proteins or enzymes which precede PKCθ in the reaction pathway (the enzyme cascade) to be modulated by the test substance, so that the modulating effect of the test substance in this reaction pathway has only an indirect effect on the activity of PKCθ.

A PKCθ “modulator” in the context of the description comprises both an activator and an inhibitor of PKCθ. Because of the function of PKCθ in T cells, on the one hand activators of PKCθ can act as immunostimulants, and on the other hand inhibitors of PKCθ can act as immunosuppressants.

“Modulation” means in the context of the description that a difference is observed in the presence of the PKCθ modulator (or of the test substance) compared with the absence of the PKCθ modulator (or of the test substance) under conditions which are otherwise identical. The modulating effect, which may be activating or inhibiting, becomes manifest in this relative comparison.

In the method of the invention, steps (a), where appropriate (b), (c), where appropriate (d) and (e) take place in the sequence in which they are mentioned, it being possible for individual steps to be carried out simultaneously. Steps (b) and (d) are optional. The method of the invention particularly preferably includes all steps (a) to (e), with preferably steps (b) and (c) being carried out simultaneously.

In the method of the invention, PKCθ serves as substrate of the phosphorylation reaction in step (c). The phosphorylation of the at least one serine or threonine residue of PKCθ can be catalysed in vivo by various kinases. The phosphorylation of the at least one serine or threonine residue of PKCθ is preferably catalysed by PKCθ itself, i.e. it proceeds at least partly as autophosphorylation.

Suitable phosphorylation sites in the method of the invention are the hydroxyl groups of serine or threonine residues of PKCθ which are phosphorylated under in vivo conditions, where appropriate after activation. In order for it to be possible to investigate an influence of the test substance to be investigated on the phosphorylation content of these serine and/or threonine residues, it is preferred for the phosphorylation of the at least one serine or threonine residue by the cell to take place at least partly only after the cell has been contacted with the test substance. This can be achieved for example by inducing the phosphorylation activity of the cell, preferably the kinase activity of PKCθ, by suitable means only after the cell has been contacted and incubated with the test substance to be investigated.

The at least one serine or threonine residue is preferably a residue which is at least partly phosphorylated with catalysis by PKCθ itself, i.e. an autophosphorylation site. It is known that there is autophosphorylation in PKCθ of the serine side chains in the turn motif at Ser⁶⁷⁶ and in the hydrophobic motif at Ser⁶⁹⁵ (cf. Y. Liu et al., Biochem. J. (2002) 361, 255-265). In contrast thereto, the tyrosine side chain in the regulatory domain at Tyr⁹⁰ is phosphorylated not by PKCθ itself but by Lck (cf. WO 01/48236). The threonine side chain in the catalytic domain at Thr⁵³⁸ is also phosphorylated not by PKCθ itself but by PDK-1.

It is particularly preferred in the method of the invention to measure the Thr²¹⁹ phosphorylation content of PKCθ. It has surprisingly been found that PKCθ has a threonine residue in the regulatory domain at Thr²¹⁹, which is phosphorylated. It was possible to confirm by phosphopeptide mapping (cf. B. D. Manning et al., Sci. STKE, 2002, 162, 49) and biochemical investigations that this involves an autophosphorylation site.

Determination of the Thr²¹⁹ phosphorylation content thus provides direct information about the enzymic activity of PKCθ in vivo.

The Thr²¹⁹ autophosphorylation site has the advantage that the hydroxyl group in the side chain of the threonine residue is very suitable, because of its position within the tertiary structure of PKCθ, as substrate for the autophosphorylation reaction catalysed by PKCθ, and is converted with a satisfactory catalytic constant. In addition, the reaction product, i.e. the phosphorylated threonine residue in position 219 of PKCθ, is also readily accessible as part of an epitope for phospho-specific antibodies, thus simplifying determination of the phosphorylation content.

Moreover, the autophosphorylation—in contrast to Ser⁶⁷⁶ and Ser⁶⁹⁵, the two other known autophosphorylation sites of PKCθ—takes place only after PKCθ has been activated. Thr²¹⁹ is therefore particularly advantageously suitable as autophosphorylation site for the method of the invention, because the kinase activity of PKCθ and thus the auto-phosphorylation at Thr²¹⁹ can be induced at a defined time. Targeted inducibility of the phosphorylation of Thr²¹⁹ at a defined time is a substantial advantage of this autophosphorylation site of PKCθ compared with the other known autophosphorylation sites.

“Phosphorylation content” means in the context of the description the molar proportion of the PKCθ molecules which is in phosphorylated form at the relevant at least one serine or threonine residue at a defined time relative to the totality of all PKCθ molecules which are phosphorylated and unphosphorylated at the relevant at least one serine or threonine residue in the system at the same time. The phosphorylation content can be reported in mol %. It is possible by recording dose-effect curves to measure the inhibition or activation of the test substance to be investigated, which is normally reported as the IC₅₀ or as a function of the concentration of the test substance in %.

Unless defined otherwise, all the technical and scientific terms used in the description have the generally customary meaning from the viewpoint of the skilled person. Concerning details and definitions of terms, reference can be made for example in their entirety to B. Alberts et al., Molecular Biology of the Cell, John Wiley & Sons; D. Voet et al., Biochemistry, John Wiley & Sons; L. Stryer et al., Biochemistry, W. H. Freeman & Company; and D. Nelson et al., Lehninger Principles of Biochemistry, Palgrave Macmillan.

In the method of the invention, in step (a) the cell is contacted with the test substance whose modulating effect is to be investigated, or with the PKCθ modulator. Depending on the cell type used, the incubation media used according to standard protocols are suitable therefor. If the cell is a human T cell, preferably a primary or murine human T cell, an example of a suitable medium is RPMI, 10% FCS, 2 mM L-glutamine, 50 u/ml penicillin/streptomycin.

The test substance to be investigated, or the PKCθ modulator, is in this case contacted with the cell in a concentration sufficient to enable detection—in the case of a modulating effect—of a difference in the phosphorylation content of the at least one serine or threonine residue of PKCθ compared with a negative control. The concentration used for the test substance or the PKCθ modulator does not depend on the number of cells used per measurement. The method of the invention is preferably carried out with from 10³ to 10⁷ cells for a test substance or for a PKCθ modulator.

Standard protocols and reagents suitable for manipulating PKCθ and cells containing PKCθ are known to the skilled person. It is possible in this connection to refer for example to R. Brent et al., Current Protocols in Molecular Biology, John Wiley & Sons Inc; J. Sambrook et al., Molecular Cloning: A Laboratory Manual, Cold Spring Harbor Laboratory; A. D. Reith, Protein Kinase Protocols, Humana Press; A. C. Newton et al., Protein Kinase C Protocols (Methods in Molecular Biology (Clifton, N.J.) V. 233.), Humana Press; J. N. Abelson et al., Protein Phosphorylation, Part A: Protein Kinases: Assays, Purification, Antibodies, Functional Analysis, Cloning, and Expression: Volume 200: Protein Phosphorulation Part A (Methods in Enzymology), Academic Press; J. F. Kuo, Proteinkinase C, Oxford University Press; and G. Hardie et al., Protein Kinase Facts Book (Two-Volume Set), Academic Press in their entirety.

In step (b) of the method of the invention there is preferably induction of the kinase activity of PKCθ. Suitable substances for activating PKCθ are known to the skilled person.

In a preferred embodiment, step (b) takes place with the aid of anti-CD3 antibodies which are preferably immobilized on beads. Suitable anti-CD3 antibodies are obtainable for example from Janssen Cilag under the designation Orthoclone OKT®3 and may be immobilized for example on beads which are marketed by Dynal Biotech Ltd. under the designation Dynabeads® Pan Mouse IgG (solid phase CD3). In this case, PKCθ is activated indirectly via the T-cell receptor (TCR).

In a preferred embodiment, step (b) takes place with the aid of direct activators. Suitable examples are diacylglycerol, bryostatins or commercially available phorbol esters such as, for example, 4α-phorbol 12-myristate 13-acetate (PMA), which have a direct effect on the kinase activity of PKCθ. The cascade via the T-cell receptor and other kinases is bypassed in this way. Accordingly, the kinase activity in step (b) is preferably induced by adding a phorbol ester, bryostatin or anti-CD3 antibody.

The induction of the activation of PKCθ which is carried out where appropriate in step (b) preferably does not take place immediately after step (a) has been carried out. On the contrary, the cell is preferably incubated after step (a), i.e. the contacting with the test substance to be investigated or with the PKCθ modulator, for a certain time which may be for example in the region of one hour. Longer or shorter incubation times are, however, also possible according to the invention. Shorter incubation times, for example of the order of from 20 to 40 minutes, are preferred.

In step (c) of the method of the invention, the cell is incubated under conditions which bring about phosphorylation of at least one serine or threonine residue of PKCθ. Only some particular serine or threonine residues of the totality of all serine and threonine residues of PKCθ are reactive and are available as substrates for phosphorylation within the cell. Preference is given in this connection to Thr²¹⁹ of PKCθ.

For this purpose, the cell is incubated at a temperature of 37° C. preferably for a time of from 1 to 30 min, more preferably 2 to 10 min. The cell is preferably incubated for a time required in the absence of the test substance to be investigated or of the PKCθ modulator, under conditions which are otherwise identical, for phosphorylation of at least 10% of the at least one serine or threonine residue, more preferably at least 15%, even more preferably at least 20% of the at least one serine or threonine residue. The time necessary for this can be found by simple preliminary tests.

If the method of the invention includes step (b), i.e. induction of the kinase activity of PKCθ, then step (c) preferably takes place under the same conditions as step (b).

Steps (b) and (c) are particularly preferably carried out simultaneously. If induction of the kinase activity of PKCθ includes for example addition of a phorbol ester, then the phorbol ester preferably remains in the incubation medium while step (c) is being carried out. Thus, there is preferably continuous activation of PKCθ through the presence of the phorbol ester (step (b)), and at the same time conditions bringing about phosphorylation of the at least one serine or threonine residue of PKCθ (step (c)) are created.

However, it is also possible to stop the induction of the kinase activity of PKCθ in step (b) before or during step (c). This can be achieved for example in step (b) through the use of anti-CD3 antibodies which are immobilized on magnetic particles and are removed from the cell or cells before step (c) is complete.

However, it is preferred for the induction of the kinase activity of PKCθ in step (b) to take place throughout step (c).

If the method of the invention includes steps (a), (b) and (c), then the cell is preferably incubated after the contacting with the test substance to be investigated or with the PKCθ modulator in step (a) for a certain time, e.g. for one hour, before the kinase activity of PKCθ is induced in step (b), and the cell is incubated in step (c) under conditions which bring about the phosphorylation of at least one serine or threonine residue of PKCθ.

In step (d) of the method of the invention, the cell is preferably lysed. The generally customary methods according to standard protocols are suitable for the lysis. Osmotic lysis or the use of surfactants such as, for example, Triton or Tween in suitable buffers are preferred. A suitable lysis buffer has for example the following composition: 50 mM Tris-HCl (pH 8.0), 100 mM NaCl, 2% Nonidet P-40, 1 mM phenylmethylsulphonyl fluoride, 0.5 μg of leupeptin per ml, and 1.0 μg of aprotinin per ml and 5 mM sodium orthovanadate. Another suitable lysis buffer consists of 50 mM HEPES (pH 7.5), 2% Nonidet P-40, 5 mM sodium orthovanadate, 5 mM sodium pyrophosphate, 5 mM NaF, 5 mM EDTA, 50 mM NaCl and 50 μg/ml aprotinin and leupeptin.

The phosphorylation content of the at least one serine or threonine residue of PKCθ is determined in step (e) of the method of the invention. The customary methods according to standard protocols are suitable in principle for this. In this connection, reference may be made for example to A. C. Newton et al., Protein Kinase C Protocols (Methods in Molecular Biology (Clifton, N.J.), V. 233.), Humana Press; J. N. Abelson et al., Protein Phosphorylation, Part A: Protein Kinases: Assays, Purification, Antibodies, Functional Analysis, Cloning, and Expression: Volume 200: Protein Phosphorulation Part A (Methods in Enzymology), Academic Press, in their entirety.

It is possible for example in step (c) to achieve a radiolabelling of the at least one serine or threonine residue by adding [³²P]-γ-ATP to the incubation medium, and to quantify the radioactivity after lysis in step (d) and isolation of the labelled PKCθ from the lysate by scintillation counting in step (e). However, since the radiolabelling is nonspecific in relation to the individual phosphorylation sites, is substantially unsuitable for HTS approaches and requires special safety precautions, the phosphorylation content of the at least one serine or threonine residue of PKCθ is preferably measured with the aid of colorimetric, fluorometric or luminometric methods. Accordingly, step (e) preferably includes a colorimetric, fluorometric or luminometric measurement.

Fluorometric methods include besides conventional fluorescence measurements also fluorescence resonance energy transfer measurements (FRET), it being possible when two fluorophores (donor and acceptor) are used for both the fluorescence quenching of the donor and the fluorescence of the acceptor to be measured.

Luminometric methods include measurement of the electrochemoluminescence. Measurement with the aid of an Amplified Luminescent Proximity Homogeneous Assay (ALPHA)Screen® (BioSignal Packard, Inc.) is also suitable.

In a preferred embodiment of the method of the invention, step (e) includes the use of ELISA technology (ELISA=enzyme-linked immunosorbent assay). ELISA technology is familiar to the skilled person. In this connection, reference may be made for example to J. R. Crowther et al., The ELISA Guidebook, Humana Press; J. R. Crowther, ELISA: Theory and Practice, Humana Press; and D. M. Kemeny, A Practical Guide to Elisa, Pergamon, in their entirety.

An enzyme-coupled immunodetection (ELISA) normally includes the following steps:
(i) the antibody against the protein which is sought (capture antibody), in this case preferably an anti-PKCθ-phospho-Thr²¹⁹ antibody, is tethered to an inert solid phase such as, for example, polystyrene;
(ii) the solution of the protein to be investigated is loaded onto the surface occupied by antibodies, so that the immobilized antibody can bind the protein;
(iii) the resulting antibody-protein complex is incubated with a second protein-specific antibody (detection antibody), in this case preferably an anti-PKCθ antibody; this second antibody is preferably covalently linked to an easily detectable enzyme (antibody-enzyme conjugate);
(iv) the excess, unbound second antibody is removed by repeated washing. The enzyme of the capture antibody-protein detection antibody-enzyme complex is then detected, from which the amount of the bound protein can be calculated.

The tethering in step (i) can be achieved in various ways. The different possibilities are familiar to the skilled person. For example, the tethering can be achieved
by solid phases which themselves are covalently linked to antibodies, these covalently linked antibodies being specific against antibodies of the organisms which were used to prepare the capture antibody;
by solid phases which are covalently linked to streptavidin or biotin, and the capture antibody in turn is conjugated to biotin or streptavidin, respectively; or
by solid phases which have on their surface suitable functional groups able to form, where appropriate after chemical activation, covalent bonds with the functional groups of the capture antibody; in this connection, reference may be made for example to M. Nisnevitch et al., J. Biochem. Biophys. Methods. 2001; 49(1-3):467-80 in its entirety.

In another preferred embodiment of the method of the invention, step (e) includes the use of FLISA technology (FLISA=fluorescence-linked immunosorbent assay). FLISA technology is familiar to the skilled person. In this connection, reference may be made for example to E. E. Swartzman et al., Anal. Biochem. 1999, 271(2), 143-51; and P. Oelschlaeger et al., Anal. Biochem. 2002, 309(1), 27-34, in their entirety.

FLISA technology differs from ELISA technology in that it is possible to dispense with washing steps, and only a single incubation step is necessary. FLISA technology is therefore particularly suitable for high-throughput screening.

In a preferred embodiment, a fluorophore-linked immunodetection (FLISA) normally includes the following steps:
(i) the antibody against the protein which is sought (capture antibody), in this case preferably an anti-PKCθ-phospho-Thr²¹⁹ antibody, is tethered to beads of an inert material (cf. above);
(ii) the solution of the protein to be investigated, and a second protein-specific antibody (detection antibody), in this case preferably an anti-PKCθ antibody, is incubated with the beads to form a capture antibody-protein-detection antibody complex. For this complex to be fluorometrically detectable, it is necessary for at least one suitable fluorophore to be present. This can be achieved in various ways. For example
   the second antibody can be covalently linked directly to a fluorophore;
   the second antibody can be conjugated with biotin, and a fluorophore bound to streptavidin can additionally be added during the incubation;
   the fluorophore can be bound to a third antibody which is added during the incubation and is specific for antibodies of the species used to prepare the detection antibody;
(iii) preferably without washing steps, the fluorescence of the capture antibody-protein-detection antbody-fluorophore complex is detected, from which the amount of bound protein can be calculated. In the measurement of the fluorescence, suitable methods are used to measure only the fluorescence of the fluorophore bound in the complex, but not the fluorescence of the excess fluorophore which is present freely in solution; this can be achieved for example
   with the aid of hydrodynamic focusing (flow cytometry), in which case the beads are passed singly and in approximately the same alignment passed a laser focus, and/or
   by labelling the beads with a second fluorophore, in which case the measurement of the fluorescence is then based on a colocalization of the two fluorescence signals.

The determination of the phosphorylation in step (e) is preferably based on the use of phospho-specific antibodies against the at least one serine or threonine residue of PKCθ which has been phosphorylated after step (a), i.e. after contacting the cell with the test substance to be investigated or with the PKCθ modulator, in step (c).

An antibody which is directed against phosphorylated threonine and whose epitope is substantially confined to the phosphorylated threonine residue and is thus substantially independent of the structure of the flanking amino acid residues is obtainable for example from New England Biolabs, Inc., Herts, GB. However, this antibody is not specific for a phosphorylated threonine residue in position 219 of PKCθ, but always binds to every phosphorylated threonine residue in any protein in the cell lysate. Since this antibody distinguishes neither between PKCθ and other proteins nor between individual phosphorylated threonine residues, its selectivity/sensitivity is correspondingly low.

Step (e) of the method of the invention preferably includes the use of an antibody which is specific against a phosphorylated threonine residue in position 219 of PKCθ, also referred to as “anti-PKCθ-phospho-Thr²¹⁹ antibody” for the purpose of the description. However, it is also possible in principle to use an antibody which binds to any phosphorylated threonine residues, also referred to as “anti-phospho-Thr antibody” for the purpose of the description.

For the purpose of the description, the notation “phospho-Thr²¹⁹” means an L-threonine residue in position 219 within the primary structure of PKCθ, whose hydroxyl group in the side chain is monophosphorylated. If the cell(s) employed in the method of the invention is/are human cells, the term “phospho-Thr²¹⁹” preferably means a phosphorylated threonine residue in position 219 within the sequence depicted as SEQ. ID. NO. 1.

These antibodies may be monoclonal or polyclonal. Suitable methods for preparing such antibodies are known to the skilled person. In this connection, reference may be made for example to E. Liddell et al., Antikörper-Techniken, Spektrum Akademischer Verlag; R. Kontermann et al., Antibody Engineering, Springer, Berlin; E. Harlow et al., Using Antibodies—A Laboratory Manual, Cold Spring Harbor Laboratory Press; E. Harlow et al., Antibodies: A Laboratory Manual, Cold Spring Harbor Laboratory Press; B. K. C. Lo, Antibody Engineering: Methods and Protocols (Methods in Molecular Biology), Humana Press; P. S. Shepherd et al., Monoclonal Antibodies: A Practical Approach, Oxford University Press; G. Subramanian, Antibodies Production and Purification, Kluwer Academic/Plenum Publishers; T. Clackson et al., Phage Display: A Practical Approach (The Practical Approach Series, 266), Oxford University Press; and B. K. Kay, Phage Display of Peptides and Proteins: A Laboratory Manual, Academic Press, in their entirety.

The primary structure of PKCθ varies depending on the organism used. It is possible to employ in the method of the invention for example the PKCθ from mice, rats or other animals. It is preferred to employ in the method of the invention human cells, preferably T cells, in particular human T cells, so that the investigated PKCθ is preferably human PKCθ. The enzyme investigated is preferably the PKCθ I isoform. The investigated PKCθ preferably includes SEQ. ID. NO. 1.

Phospho-specific antibodies against particular phosphorylation sites of PKCθ are prepared preferably by synthesizing oligopeptides whose primary structure corresponds to the region around the phosphorylation site within the primary structure of PKCθ. A phospho-specific antibody against Thr²¹⁹ can therefore be prepared for example with the aid of an oligopeptide including the partial sequence . . . Glu-(phospho-Thr)-Met . . . , where “phospho-Thr” represents a threonine residue phosphorylated in the side chain. Suitable methods for preparing such oligopeptides are known to the skilled person. In this connection, reference may be made for example to M. W. Pennington et al., Peptide Synthesis Protocols (Methods in Molecular Biology), Humana Press, 1994; W. C. Chan et al., Fmoc Solid Phase Peptide Synthesis: A Practical Approach, Oxford University Press, 2000; J. Jones, Amino Acid and Peptide Synthesis (Oxford Chemistry Primers, 7), Oxford University Press, 2002; J. Howl, Peptide Synthesis And Applications (Methods in Molecular Biology), Humana Press, 2005; and N. L. Benoiton, Chemistry of Peptide Synthesis, CRC Press, 2005.

An anti-PKCθ-phospho-Thr²¹⁹ antibody is prepared preferably by using an oligopeptide which includes an amino acid sequence of at least 5 amino acid residues, preferably at least 7, more preferably at least 9, even more preferably at least 11, most preferably at least 13 and especially at least 15 amino acid residues, with the proviso that this amino acid sequence corresponds to a continuous partial sequence of SEQ. ID. NO. 2 and moreover includes the phosphorylated threonine residue which has position 19 in SEQ. ID. NO. 2. The amino acid sequence preferably includes positions 17 to 21, more preferably 15 to 23, even more preferably 13 to 25, most preferably 11 to 27 and especially 9 to 29 of SEQ. ID. NO. 2.

The oligopeptide can subsequently be conjugated for example with maleimide-activated keyhole limpet haemocyanin (KLH) or bovine serum albumin (BSA). It is possible thereafter to immunize a plurality of individuals, for example white New Zealand rabbits, with the peptide-KLH conjugate, the immunization being repeated at regular intervals, for example after 2 weeks. The antibody titre in the serum can be determined by an ELISA in peptide-BSA-coated microtitre plates. The phospho-specific antibodies, in this case preferably anti-PKCθ-phospho-Thr²¹⁹ antibodies, can then be isolated from the serum by suitable methods.

Monoclonal antibodies can be prepared analogously by preparing hybridomas, for example with the aid of immunized mice, i.e. monoclonal anti-PKCθ-phospho-Thr²¹⁹ antibodies. For this purpose, following the immunization, the antibody-forming B lymphocytes are isolated, preferably from the spleen of mice, and subsequently fused with myeloma cells, resulting in hybridoma cells. It is also possible to obtain monoclonal antibodies from rabbits (RabMab; cf., for example, H. Spieker-Polet et al., Proc. Natl. Acad. Sci. 1995 Sep. 26; 92(20): 9348-52). The Phage Display technology can also be used to generate monoclonal antibodies (cf. for example P. G. Schultz et al., Science 1995, 269: 1835-1842).

The polyclonal or monoclonal antibodies obtained in this way can furthermore be conjugated with fluorescent dyes, enzymes, biotin, etc., and/or be immobilized on solid phases. The method steps necessary for this take place in accordance with standard protocols.

The method of the invention preferably includes in step (e) the following substeps:
(e₁) immunoprecipitation of at least part of the PKCθ using a suitable first antibody; and
(e₂) measurement of the phosphorylation content of the at least one serine or threonine residue of the immunoprecipitated PKCθ by using a suitable second antibody.

It is preferred in this connection for the first antibody to be directed against PKCθ (ant-PKCθ antibody) and for the second antibody to be directed against phospho-Thr²¹⁹ of PKCθ (anti-PKCθ-phospho-Thr²¹⁹ antibody), or vice versa.

Preferred embodiments of step (e) of the method of the invention are described below:

### Western Blotting:

In a preferred embodiment of the method of the invention, the lysis (step (d)) is followed by an immunoprecipitation of the PKCθ from the lysate using an anti-PKCθ antibody (Ab1), which is preferably monoclonal. Ab1 is preferably coupled to a support matrix, for example to protein G Sepharose.

The precipitate is then divided preferably into two portions which are preferably of equal size. The PKCθ present in the precipitate is then separated from each of the other constituents, preferably by gel electrophoresis (1D-SDS-PAGE), and transferred to a membrane by Western Blotting.

The phosphorylation of the phosphorylation site, preferably Thr²¹⁹, in one of the two samples is detected with the aid of a suitable phospho-specific antibody (Ab2), preferably with the aid of an anti-PKCθ-phospho-Thr²¹⁹ antibody.

On the other hand, the total amount of precipitated PKCθ, i.e. of phosphorylated and unphosphorylated PKCθ, is detected in the other sample as loading control. It is possible to use for this purpose for example the anti-PKCθ antibody (Ab1).

The resulting bands are preferably evaluated by densitometry, with the phospho signal being normalized to the respective total amount of PKCθ (loading control). The evaluation by densitometry preferably takes place with the aid of anti-PKCθ antibodies or antibodies against anti-PKCθ antibodies conjugated with the usual enzymes which, after addition of suitable substrates, catalyse a colour reaction or a chemoluminescence reaction. Examples of suitable enzymes are alkaline phosphatase, horseradish peroxidase (HRPO), β-galactosidase, glucoamylase, glucose oxidase and luciferase. A monoclonal anti-PKCθ antibody which is conjugated to horseradish peroxidase (HRPO) is commercially available for example from BD Biosciences Pharmingen, San Diego, USA.

The anti-PKCθ antibody can also be conjugated directly to a fluorescent dye, for example to AMCA, Cy3, Cy5, fluorescein, Hoechst 33258, B-phycoerythrin, R-phycoerythrin, rhodamine or Texas Red®. Normalization of the measurements preferably takes place by calibration of the method. A recombinant phosphomutant can in this case be used as negative control, and recombinant PKCθ already phosphorylated on Thr²¹⁹ as positive control.

In a preferred embodiment, the sample is not divided into two parts and analysed in two separate Western Blots, but is analysed completely and simultaneously on a single Western Blot For this purpose, the anti-PKCθ antibody (Ab1) and the phospho-specific antibody (Ab2) are preferably prepared with the aid of different species, so that a species-specific differentiation is possible when evaluating the bands: if, for example, Ab1 has been obtained by immunizing rabbits and Ab2 by immunizing mice, it is possible to add for the evaluation two fluorophores F1 and F2, of which one is conjugated to an anti-mouse antibody and the other to an anti-rabbit antibody. In this way, both fluorescence signals can be evaluated on the same Western Blot.

Dose-effect curves can be generated by a plurality of measurements at different concentrations of the test substance to be investigated.

### Elisa:

In another preferred embodiment of the method of the invention, lysis (step (d)) is followed by determination of the phosphorylation content of the at least one serine or threonine residue of PKCθ by using ELISA technology. In this case, an anti-PKCθ antibody and an anti-PKCθ-phospho-Thr²¹⁹ antibody are preferably used in a sandwich ELISA. For this purpose, preferably one of the two antibodies is immobilized on the inner surface of the wells of a microtitre plate. An anti-PKCθ-phospho-Thr²¹⁹ antibody is preferred in this connection. This antibody is preferably used as primary antibody (“capture antibody”).

The lysate obtained in step (d) in the method of the invention is then put into the wells of the microtitre plates. An incubation time is preferably followed by a plurality of washing steps.

The second antibody is then added, this preferably being an anti-PKCθ antibody, preferably monoclonal. This antibody serves as secondary antibody (“detection antibody”). Detection of the binding complex of the phosphorylated PKCθ (antigen) and the two antibodies can then take place with the aid of calorimetric or fluorometric or luminometric methods.

For this purpose, the secondary antibody can be conjugated for example with one of the usual enzymes which subsequently, after addition of suitable substrates, catalyse a colour reaction or a chemoluminescence reaction. Examples of suitable enzymes are the aforementioned enzymes.

The enzyme may also be conjugated to streptavidin and be bound to a biotinylated secondary antibody which is in turn conjugated with one of the aforementioned enzymes. Signal enhancement is possible if the molar ratio of biotin to secondary antibody and/or enzyme to streptavidin is >1.

The detection antibody may also be conjugated directly to a fluorescent dye. Examples of suitable fluorescent dyes are mentioned above. The measurements are normalized preferably by calibration of the method. A recombinant phosphomutant can in this case be used as negative control, and recombinant PKCθ which is already phosphorylated on Thr²¹⁹ as positive control.

### FLISA:

In another preferred embodiment of the method of the invention, the lysis (step (d)) is followed by determination of the phosphorylation content of the at least one serine or threonine residue of PKCθ by using FLISA technology. Since washing steps are usually impossible or can be achieved only in an elaborate fashion in high-throughput screening (HTS) systems, this particularly preferred method preferably takes place by use of an antibody which is immobilized on beads.

For this purpose, preferably a first antibody (Ab1), preferably an anti-PKCθ-phospho-Thr²¹⁹ antibody, is immobilized as primary antibody on beads which are labelled with a first fluorescent dye (F1).

After lysis of the cell(s) in step (d), the lysates are incubated with these beads. Subsequently, a second antibody (Ab2), preferably an anti-PKCθ antibody, is added as secondary antibody.

In a preferred embodiment, evaluation takes place by hydrodynamic focusing (flow cytometry), for example with the aid of a BD-FACSArray®Bicanalyzer from BD Biosciences. Only a single fluorescent dye is necessary for this. The procedure for the evaluation takes place in accordance with standard protocols and is familiar to the skilled person.

In another preferred embodiment, the secondary antibody (Ab2) is labelled with a second fluorescent dye (F2), so that two different fluorescent dyes are present in the system. The bead complex is then preferably detected in a confocal system (e.g. using an Opera reader from Evotec OAI AG, Hamburg, Germany). In a variant of this embodiment, the secondary antibody (Ab2) is conjugated with biotin, and the second fluorescent dye (F2) is prepared as conjugate with streptavidin, so that it is able to bind to the biotinylated secondary antibody (Ab2).

Evaluation based on FLISA technology has the advantages that all the steps from the contacting of the cell with the test substance to be investigated up to measurement of the fluorescence can be carried out in the same microtitre plate. Washing steps can be dispensed with owing to the confocal measuring technique.

If the measurement is based on the use of two fluorophores, the results of measurement are assessed as positive only in the event of colocalization of the two fluorescence signals (F1+F2). The use of two antibodies and of two fluorescence markers therefore increases the specificity. Examples of suitable fluorescent dyes F1 and F2 are the following pairs: F1:R-phycoerythrin, Cy3 (Alexa® 532) F2:APC, Cy5, Alexa® 647, Alexa® 633.

Alternatively, the measurement can take place on the laboratory scale also in a flow cytometer or, for example, using the Luminex reader from Luminex Corporation, Austin, USA. Evaluation by fluorescence resonance energy transfer measurements (FRET) is also possible.

Accordingly, the method of the invention preferably includes in step (e) the use of ELISA or FLISA technology. The method particularly preferably includes in step (e) the use of FLISA technology, in which case two different fluorescent dyes are used, and the measurement of the phosphorylation content is based on the measurement of the fluorescence of the two dyes.

In a preferred embodiment, the method of the invention includes the further step
(f) comparison of the phosphorylation content of the at least one serine or threonine residue of PKCθ which has been determined in step (e) with the corresponding phosphorylation content which is determined when the method is carried out under conditions which are otherwise identical but without step (a), i.e. in the absence of the test substance or of the PKCθ modulator.

The method of the invention is suitable for investigating the modulating effect of a test substance or of a PKCθ modulator on a PKCθ-dependent signal transduction pathway in a human or animal cell.

The test substances or PKCθ modulators which can be found in the method of the invention are suitable for the prevention and/or treatment of PKCθ-mediated diseases. The method can therefore be used in the search for novel pharmacological active ingredients, especially novel immunomodulators, such as immunostimulants and immunosuppressants, but also novel agents for treating muscle disorders.

Immunostimulants are increasingly being employed for assisting the patients' biological response to tumours. This can take place for example by strengthening the immune response. The cytotoxicity of T cells and, where appropriate, also the activity of natural killer cells can be increased by these substances. Immunostimulants are also employed in the treatment of chronic hepatitis C and of HIV. Some immunostimulants are also employed for the prophylaxis of colds.

Immunosuppressants are suitable for the treatment of various indications, for example
for the treatment of acute or chronic inflammatory processes and inflammatory disorders (for example inflammatory airway disorders such as COPD [chronic obstructive pulmonary disease], asthma, etc.),
for the treatment of allergies (for example the severe anaphylactic immediate reaction, etc.),
for the treatment of autoimmune diseases (for example rheumatoid arthritis, Crohn's disease, ulcerative colitis, uveitis, psoriasis, nephrotic syndrome, diabetes 1, diabetes 2, multiple sclerosis, etc.)
for the treatment of septic shock,
for the prophylaxis or therapy of ischaemia/reperfusion damage (e.g. myocardial infarction, stroke, etc.) and
for the prophylaxis or therapy of the rejection response after a transplant (for example of kidney, liver, heart, lung, pancreas, lens of the eye, bone marrow, etc).

A further aspect of the invention relates to an antibody against a phosphorylated threonine residue in position 219 of PKCθ (anti-PKCθ-phospho-Thr²¹⁹ antibody). This antibody may be polyclonal or monoclonal. The antibody in this case is one which is specific for a phosphorylated threonine residue in position 219 of PKCθ, i.e. it is not a nonspecific anti-phospho-Thr antibody which also binds to phosphorylated threonine residues which are not flanked by the same amino acids as Thr²¹⁹ of PKCθ.

The anti-PKCθ-phospho-Thr²¹⁹ antibody preferably has an affinity constant for Thr²¹⁹ of PKCθ of less than 10⁻⁴ M, more preferably of less than 10⁻⁵ M, even more preferably of less than 10⁻⁶ M, most preferably of less than 10⁻⁷ M and in particular of less than 10⁻⁸ M or even less than 10⁻⁹ M. Suitable for determining the affinity constant is for example surface plasmon resonance spectroscopy (e.g. using an instrument from Biacore, Neuchatel, Switzerland).

The anti-PKCθ-phospho-Thr²¹⁹ antibody of the invention is specific for Thr²¹⁹ of PKCθ, i.e. it binds to a phosphorylated threonine residue in position 219 but not to any of the other threonine residues of PKCθ, if phosphorylated. Binding of the anti-PKCθ-phospho-Thr²¹⁹ antibody of the invention to PKCθ thus depends on the structure of the amino acid residues which flank the phosphorylated threonine residue in position 219 of PKCθ. The anti-PKCθ-phospho-Thr²¹⁹ antibody of the invention in particular does not include an anti-phospho-Thr antibody which binds to any phosphorylated threonine residues, irrespective of the sequence of the flanking amino acid residues.

Thus, the anti-PKCθ-phospho-Thr²¹⁹ antibody of the invention is specific for an epitope which includes more than the phosphorylated threonine residue. Examples of such epitope substructures are -Glu²¹⁸-Thr²¹⁹-, -Thr²¹⁹-Met²²⁰-, and -Glu²¹⁸-Thr²¹⁹-Met²²⁰-, etc. The epitope preferably includes an amino acid sequence of at least 5 amino acid residues, preferably at least 7, more preferably at least 9, even more preferably at least 11, most preferably at least 13 and in particular at least 15 amino acid residues, with the proviso that this amino acid sequence corresponds to a continuous partial sequence of SEQ. ID. NO. 2 and moreover includes the phosphorylated threonine residue which has position 19 in SEQ. ID. NO. 2. The epitope preferably includes the partial sequence of positions 17 to 21, more preferably 16 to 22, even more preferably 15 to 23, most preferably 14 to 24 and especially 13 to 25 of SEQ. ID. NO. 2. In this connection, “specific” means that the antibody does not bind to an epitope which does not include the abovementioned partial sequence, although including a phosphorylated threonine residue.

In a preferred embodiment, the anti-PKCθ-phospho-Thr²¹⁹ antibody of the invention is a polyclonal antibody. In another preferred embodiment, the anti-PKCθ-phospho-Thr²¹⁹ antibody of the invention is a monoclonal antibody which can preferably be produced by a hybridoma cell line as RabMab or Phage Display.

A further aspect of the invention relates to a method for preparing an anti-PKCθ-phospho-Thr²¹⁹ antibody described above, including the injection of an oligopeptide (antigen) into a suitable organism, e.g. rabbit or mouse, where the oligopeptide includes an amino acid sequence of at least 5 amino acid residues, preferably at least 7, more preferably at least 9, even more preferably at least 11, most preferably at least 13 and in particular at least 15 amino acid residues, with the proviso that this amino acid sequence corresponds to a continuous partial sequence of SEQ. ID. NO. 2 and moreover includes the phosphorylated threonine residue which has position 19 in SEQ. ID. NO. 2.

The oligopeptide preferably includes the partial sequence of positions 17 to 21, more preferably 16 to 22, even more preferably 15 to 23, most preferably 14 to 24 and in particular 13 to 25 of SEQ. ID. NO. 2.

The oligopeptide is moreover preferably conjugated before the immunization to a suitable carrier protein, for example to KLH. Suitable kits for conjugation of antigens to carrier proteins are commercially available. They are used in accordance with standard protocols.

The antibody can then be isolated from the plasma by conventional methods, for example by affinity chromatography.

Monoclonal anti-PKCθ-phospho-Thr²¹⁹ antibodies can be obtained from hybridoma cells of mice, from rabbits (RabMab) or by Phage Display. These methods are known to the skilled person.

In a preferred embodiment, the method of the invention for preparing an anti-PKCθ-phospho-Thr²¹⁹ antibody relates to a selection step on the basis of which specific antibodies are separated from nonspecific antibodies which are possibly present, i.e. anti-PKCθ-phospho-Thr²¹⁹ antibodies from anti-phospho-Thr antibodies. This can be achieved preferably by affinity chromatography. It is possible for this purpose for example to immobilize on the stationary phase phosphorylated threonine residues which are incorporated into a peptide sequence, with the amino acid residues which flank the phosphorylated threonine residue differing from the amino acid residues which are present in the corresponding position in the case of native Thr²¹⁹ in PKCθ. Nonspecific anti-phospho-Thr antibodies are bound to this stationary phase, whereas the desired specific anti-PKCθ-phospho-Thr²¹⁹ antibodies are eluted since a suitable binding site is lacking.

The invention also relates to an anti-PKCθ-phospho-Thr²¹⁹ antibody obtainable by this method.

A further aspect of the invention relates to the use of an anti-PKCθ-phospho-Thr²¹⁹ antibody described above for finding a test substance having a modulating effect on a PKCθ-dependent signal transduction pathway, or a PKCθ modulator, in a human or animal cell.

The following examples serve to illustrate the invention in detail but are not to be interpreted as restricting its scope.

### EXAMPLE 1

### Preparation of an Anti-PKCθ-phospho-Thr²¹⁹ Antibody

The amino acid sequence INSRE-T(p)-MFHKE which corresponds to the partial sequence of human PKCθ in positions 214 to 224 with phosphorylated Thr²¹⁹ is prepared as antigen. The amino acid sequence is coupled in accordance with a standard protocol to keyhole limpet haemocyanin (KLH) as carrier.

Rabbits are immunized intraperitoneally using complete Freund's adjuvant. The injection is repeated after 28 days, using incomplete Freund's adjuvant for this and all further repeat injections. A first serum sample of about 5 ml is taken after 35 days. The injection is repeated again after 49 and 63 days. A second serum sample of about 5 ml is taken after 70 days. The injection is repeated after 84 days. After 91 days, exsanguination by cardiac puncture on the anaesthetized animal is possible. Alternatively, the immunization is repeated at an interval of 4 weeks and a serum sample is taken one week later in each case.

The immunoglobulins are purified by affinity chromatography, the antigen previously being immobilized in the phosphorylated state used on the stationary phase for this purpose. This is followed by affinity chromatography on a stationary phase which carries an analog of the antigen (in the unphosphorylated state). The eluate is concentrated and dialysed against PBS using a stirred cell.

### EXAMPLE 2

### Densitometric Evaluation

The assay is carried out by immunoprecipitation and detection of autophosphorylation in a Western Blot. For this purpose, the dose-dependent inhibition of PKCθ autophosphorylation of Thr²¹⁹ in human T cells is investigated using the PKC inhibitors (a) Calbiochem GF 109 203X and (b) Roche Ro 31-8220.

Primary human T cells are preincubated for 1 hour in each case with the inhibitors (a) and (b) in various concentrations. This is followed by induction of autophosphorylation by PMA (100 nM) for 5 minutes.

After washing with cold PBS, the T cells are lysed on ice for 30 minutes. The lysis buffer used is a buffer of the following composition: 50 mM HEPES (pH 7.5), 2% Nonidet P-40, 5 mM sodium orthovanadate, 5 mM sodium pyrophosphate, 5 mM NaF, 5 mM EDTA, 50 mM NaCl and 50 μg/ml aprotinin and leupeptin. Insoluble fractions are removed by centrifugation at 10 000 g and 4° C. for 15 minutes.

Phosphorylation of PKCθ on Thr²¹⁹ is detected in the lysate. For this purpose, PKCθ is immunoprecipitated from the lysates using a monoclonal anti-PKCθ antibody (Ab1, from BD Transduction Laboratories, BD Biosciences) which has previously been coupled to protein G Sepharose as support matrix. Incubation takes place at 4° C. on a rotating wheel for 2 hours. After the support matrix has been washed it is mixed with Lammli sample buffer and boiled at 95° C. for 5 minutes.

The supernatant is divided into two approximately equal-sized portions which are each fractionated in 1D SDS-PAGE gel.

The first sample is incubated in a Western Blot with the anti-PKCθ-phospho-Thr²¹⁹ antibody (Ab 2) prepared as in Example 1. The autophosphorylation is determined by adding in accordance with a standard protocol α-rabbit HRPO (horseradish peroxidase) as secondary antibody.

The second sample is incubated with Ab 1 in a Western Blot. The total amount of precipitated PKCθ is then determined by adding in accordance with a standard protocol α-mouse HRPO (horseradish peroxidase) as secondary antibody as loading control.

Detection is by chemoluminescence (Lumi-Light^{Plus} Western Blotting Substrate, Roche+ECL Plus, Amersham, Software Aida). The phospho signal is moreover normalized to the total amount of PKCθ in each case (loading control).

### EXAMPLE 3

### Evaluation by FLISA

1×10⁷ Jurkart TAg cells are transfected with 5-20 μg of human recombinant PKCθ (pEFneo) (cf. Baier-Bitterlich, Mol. Cell. Biol., 1996, 16:1842). The transient transfection is carried out using the Easy-jecT Plus electroporator from Equibo (450V, 1650 μF).

The cells are treated 1 hour before the simulation with the PKCθ inhibitor GF109 203X (Calbiochem). Autophosphorylation is induced by PMA (100 nM) for 15 minutes. After washing with cold PBS, the cells are lysed on ice for 30 minutes in analogy to Example 2. Insoluble fractions are removed by centrifugation at 10 000 g and 4° C. for 15 minutes.

Liquichip® activated beads (Qiagen) are covalently coupled in accordance with a standard protocol to the anti-PKCθ-phospho-Thr²¹⁹ antibody prepared as in Example 1 as capture antibody. The beads are then incubated with the cell lysate for 2 hours at room temperature with shaking in 96-well microtitre plates in the dark.

The monoclonal anti-PKCθ antibody (Ab1, from BD Biosciences) is then added as detection antibody and shaken at room temperature for 1 hour. This is followed by shaking at room temperature with a biotinylated anti-mouse antibody (eBioscience) for 30 minutes. Streptavidin-coupled phycoerythrin (Phycolink-SAPE, Prozyme) is incubated as detection reagent while shaking at room temperature for a further 30 minutes. These steps are likewise carried out in lysis buffer.

Detection takes place with the Luminex 100 IS (Luminex Corporation, Texas) measuring instrument.

1. A method
for investigating the modulating effect of a test substance on a PKCθ-dependent signal transduction pathway or
for finding a PKCθ modulator
in a human or animal cell, comprising the steps
(a) contacting the cell with the test substance or with the PKCθ modulator;
(b) optionally inducing the kinase activity of PKCθ;
(c) incubating the cell under conditions which bring about phosphorylation at least of a serine or threonine residue of PKCθ;
(d) optionally lysing the cell; and
(e) determining the phosphorylation content of the at least one serine or threonine residue of PKCθ.

2. The method according to claim 1, characterized in that the at least one serine or threonine residue of PKCθ includes the threonine residue in position 219.

3. The method according to claim 1, characterized in that it includes the use of an antibody against a phosphorylated threonine residue in position 219 of PKCθ.

4. The method according to claim 1, characterized in that the cell is a T cell.

5. The method according to claim 1, characterized in that step (e) includes the substeps:
(e₁) immunoprecipitation of at least part of the PKCθ using a suitable first antibody; and
(e₂) determination of the phosphorylation content of the at least one serine or threonine residue of the immunoprecipitated PKCθ by using a suitable second antibody.

6. The method according to claim 5, characterized in that the first antibody is directed against PKCθ and the second antibody is directed against a phosphorylated threonine residue in position 219 of PKCθ.

7. the method according to claim 1, characterized in that the kinase activity is induced in step (b) by adding a phorbol ester or anti-CD3 antibodies.

8. The method according to claim 1, characterized in that step (e) includes a colorimetric, fluorometric or luminometric measurement.

9. The method according to claim 8, characterized in that step (e) includes the use of Western Blotting, ELISA or FLISA technology.

10. The method according to claim 9, characterized in that step (e) include the use of FLISA technology, with two different fluorescent dyes being used, and the measurement of the phosphorylation being based on measurement of the fluorescence of the two dyes.

11. The method according to claim 1, characterized in that it includes the step
(f) comparison of the phosphorylation content determined in step (e) with the corresponding phosphorylation content which is determined when the method is carried out under conditions which are otherwise identical but without step (a).

12. An antibody against a phosphorylated threonine residue in position 219 of PKCθ.

13. (canceled)

14. The method according to claim 3, characterized in that the test substance or the PKCθ modulator is an immunostimulat or an immunosuppressant.
